(19) United States
(12) Patent Application Publication
(10) Pub. No.: US 2022/0133915 A1
(43) Pub. Date: May 5, 2022

(54) HIGH YIELD PRODUCTION OF MICROBUBBLES

(71) Applicant: Advanced Microbubbles Inc., Newark, CA (US)

(72) Inventors: Jameel Feshitan, Eureka, CA (US); Steve TYRELL, Newark, CA (US)

(21) Appl. No.: 17/431,405

(22) PCT Filed: Feb. 3, 2020
(86) PCT No.: PCT/US2020/016380

§ 371 Date: Aug. 16, 2021

Related U.S. Application Data
(60) Provisional application No. 62/807,509, filed on Feb. 19, 2019.

(51) Int. Cl. A61K 49/22; B01F 23/411; B01F 23/80; B01F 31/85

(52) U.S. Cl. CPC A61K 49/223; B01F 23/4111; B01F 2101/2202; B01F 31/85; B01F 23/807

(57) ABSTRACT

Microbubble production and size isolation with high yield processing. Specifically, a size isolation process is used in which a diffusion coefficient related to gas diffusion forces acting on microbubbles in suspension is controlled through maintaining diffusion parameters for the suspension. Diffusion parameters may include effective viscosity, which may be a function of microbubble volume fraction. Another diffusion parameter controlled may include temperature. In turn, microbubbles may be size isolated at high yields, which may provide for advantageous microbubble products that demonstrate increased stability for storage.

### CROSS REFERENCE TO RELATED APPLICATIONS

This application is a U.S. National Stage of PCT/US20/16380 filed on Feb. 3, 2021, entitled “HIGH YIELD PRODUCTION OF MICROBUBBLES” which claims priority to U.S. Provisional App. No. 62/807,509 entitled “HIGH YIELD PRODUCTION OF MICROBUBBLES” filed on 19 Feb. 2019, the entirety of which is incorporated by reference herein.

### FIELD

The present disclosure generally relates to production of microbubbles for medical use and in particular to size isolation of a population of polydisperse microbubbles at high yield.

### BACKGROUND

The use of microbubbles has been proposed in a number of applications including in a variety of biomedical contexts. Microbubbles generally describe structures comprising a shell surrounding a gas core and having a size (typically expressed as a diameter) from about 0.5 μm to about 25 μm. The shell of microbubbles may be made of different materials, although one contemplated approach includes microbubbles having a lipid shell surrounding the gas core. Regardless of the microbubble type, the use of microbubbles has been explored for use in conjunction with ultrasound devices for a number of purposes. Specifically, application of energy to microbubbles (e.g., after introduction into the body) may result in excitation of the shell and/or gas core of the microbubble to, for instance, resonate the microbubble. This may result in popping of the microbubble at a selected location within the body upon selective application of energy thereto. Accordingly, microbubbles have been proposed for use as contrast agents in medical imaging, drug delivery, extravascular delivery, noninvasive surgery or other approaches. Specifically, ultrasound devices or the like may provide targeted energy delivery to excite bubbles for specific responses in a targeted area in the body.

However, drawbacks in relation to microbubble production and processing have resulted in limitations in the adoption of microbubbles. In particular, it is been recognized that size isolated microbubbles of a given microbubble target size may be useful in certain contexts. For instance, by controlling the size of microbubbles to a size isolated range, the frequency of the energy applied to microbubbles in the body may be adapted for a specific response. Current methods for microbubble production either result in a polydisperse size distribution or suffer from limitations in microbubble yield such that sufficient quantities may not be feasibly produced for many applications contemplated. In response, certain approaches have been proposed such as those described in U.S. Patent Publication No. 2011/0300078, the entirety of which is incorporated by reference herein.

However while the approaches described in these proposed approaches may produce relatively high yields of polydisperse microbubble populations, the process to isolate the target size of microbubbles may significantly reduce the resulting yield of the size isolated microbubbles. Moreover, preservation (e.g., during distribution and storage) may be difficult to achieve, thus resulting in a limited shelf life of the resultant size isolated microbubble population achieved in the size isolation process. Accordingly, drawbacks continue to exist that limit the practical application and adoption of microbubbles in a number of contexts.

### SUMMARY

In view of the foregoing, the present disclosure provides approaches including systems, methods, and products, that may facilitate high-yield production of size isolated microbubbles. Moreover, the approaches described herein that result in high-yield production of the size isolated microbubbles may further facilitate improvements in relation to preservation of size isolated microbubbles to provide unique microbubble products with good shelf stability. In turn, the present disclosure facilitates more efficient production of size isolated microbubbles that also provide benefits in relation to increase preservation, thus allowing increased practicality of microbubbles for use in a number of contexts previously limited due to the number of microbubbles capable of being produced and/or limitations in the stability of prior microbubble products.

In particular, the present disclosure leverages a recognition by the present inventor of an affect to yield during size isolation processing of the polydisperse microbubble population. For instance, centrifugation may be utilized to isolate a specific size of microbubbles from a polydisperse microbubble population. Prior approaches of centrifugation have recognized use of Stoke's equation for determination of a Relative Centrifugal Force (RCF) to target isolation of the particular size of microbubble desired. These approaches have typically been limited to volume fractions of the microbubbles in a suspension below 20%. The rationale for limiting the volume fraction of microbubbles in a suspension for application of centrifugal size sorting has been related to limitations in view of turbulence in the suspension. Specifically, volume fractions exceeding the 20% limit are not accurately modeled using Stoke's equation, thereby limiting the applicability of Stoke's equation in practice. Accordingly, size isolation using centrifugation is typically used with relatively low volume fractions of microbubbles in a separation column to which centrifugation is applied. The result has been limited yields in size isolated microbubbles after application of centrifugation.

However, the present disclosure recognizes that yields of microbubbles may be limited when a diffusion force applicable on the microbubbles in a separation column being subjected to centrifugation exceeds certain bounds. That is, with an increase in the diffusion force acting on the microbubbles during separation, an increased number of microbubbles tend to pop or otherwise degrade, which results in a limited yield. However, it is presently recognized that by reducing or minimizing a diffusion force during size isolation processing of microbubbles, the resulting yield of size isolated microbubbles may be increased as the microbubbles tend not to experience diffusion forces that degrade the microbubbles during size isolation processing. In particular, a number of diffusion factors or diffusion parameters have been identified that affect the diffusion forces experienced by microbubbles during size isolation using centrifugal force. By controlling one or more of these diffusion parameters, yields of size isolated microbubbles may be increased as compared to previously proposed techniques for size isolation.

Furthermore, it is further recognized herein that diffusion forces acting on microbubbles may also affect the stability of a resulting size isolated microbubble product. As such, controlling the diffusion parameters for a size isolated population of microbubbles may also facilitate improved stability of the size isolated microbubble product. In addition, the ability to achieve high yields during the size isolation process may assist in achieving diffusion parameters of a resultant size isolated microbubble product that is more stable. Accordingly, a number of size isolated microbubble products are described herein, each of which may leverage the recognition of the effect of diffusion forces on microbubbles to provide increased stability of a size isolated microbubble product. In particular, certain products are contemplated that may be particularly of use in relation to biomedical applications that utilize size isolated microbubbles (e.g. including certain form factors and other packaging characteristics that may be particularly advantageous in these contexts).

A number of diffusion parameters have been identified that affect the diffusion forces applicable to microbubbles during centrifugation associated with size isolation of microbubbles from a polydisperse population of microbubbles. Accordingly, any one or more of the diffusion parameters identified relating to diffusion forces applicable on microbubbles may be controlled during the size isolation process to improve yield of the process. For instance, the viscosity of the fluid containing the microbubbles may affect the diffusion forces acting on the microbubbles therein. In particular, an effective viscosity of the fluid may be of particular relevance. By effective viscosity, it is recognized that the presence of microbubbles within the fluid may affect the viscosity of the fluid in which the microbubbles are disposed. Specifically, a volume fraction of microbubbles in a fluid may affect the effective viscosity of the suspension comprising the base fluid and the microbubbles. In turn, the volume fraction of microbubbles may be particularly relevant parameter that is controlled during size isolation. In particular, it is been found that a relatively high volume fraction of microbubbles in solution advantageously affects a diffusion constant that is descriptive of the diffusion forces acting on microbubbles in a solution. Namely, volume fractions of microbubbles greater than 20% have been found to advantageously provide increased yields by increasing the effective viscosity of the fluid containing microbubbles upon which size isolation is applied. Accordingly, and in contrast to prior approaches that limit the volume fraction to at most 20%, the present approach may utilize volume fractions of microbubbles in a solution greater than 20% to advantageously control the diffusion forces acting on the microbubbles in the solution. The improvement to yields using this greater volume fraction may exceed a simple increase in yield due to the larger amount of microbubbles subjected to the process. Rather, a larger percentage of the initial microbubbles may survive size isolation processing by using the higher volume fraction as will be described in greater detail below.

Still other diffusion parameters may also be controlled to provide increased yields by reducing or minimizing the diffusion forces acting on microbubbles in solution being subjected to size isolation through centrifugation. For instance, a temperature of the suspension has been found to be a significant driver in the diffusion forces acting on microbubbles in suspension. Accordingly, a reduction in temperature or maintaining a relatively low suspension temperature may assist in reduction or minimization of the diffusion forces acting on microbubbles in the solution, thus increasing yields.

Further still, the present disclosure recognizes a dependency on the size of the targeted microbubbles in relation to the diffusion forces acting thereon. In this regard, control of the diffusion parameters that affect the diffusion forces acting upon microbubbles in the solution being subjected to size isolation may be at least in part based upon the target size of microbubbles sought during the size isolation process. In this regard, parameters for isolation of different respective target sizes of microbubbles may be controlled differently based upon the target size of microbubble sought.

A first aspect of the preset disclosure includes a method for isolation of target sized microbubbles from a polydisperse population of microbubbles at high yield. The method includes preparing a solution comprising the polydisperse population of microbubbles and maintaining a microbubble volume fraction in the solution at greater than about 20% and not greater than about 50%. In turn, the method includes applying a centrifugal field to the solution to isolate the target sized microbubbles from the solution to produce a cake of the target sized microbubbles at a target microbubble concentration of not less than about 1.0×10⁹ microbubbles(MB)/mL.

A number of feature refinements and additional features are applicable to the first aspect. These feature refinements and additional features may be used individually or in any combination. As such, each of the following features that will be discussed may be, but are not required to be, used with any other feature or combination of features of the first aspect.

For example, the method may further comprise containing the cake of the target sized microbubbles at the target microbubble concentration in a sealed container. In an embodiment, the sealed container may be a syringe. Alternatively, the sealed container may be a vial. In any regard, in an embodiment the sealed container may have an internal volume and a volume of the cake may be substantially the same as the internal volume such that no fluid headspace is present in the sealed container containing the cake.

As will be described in greater detail below, a resulting microbubble product produced from a high yield size isolation process may exhibit improved stability. In this regard, the cake of the target sized microbubbles may maintain not less than 90% of the target bubble concentration when maintained at a temperature not greater than 4° C. for a duration not less than 4 weeks from a time of containing of the cake in the container. Alternatively, improved stability may also be achieved with storage at room temperature. As such, the cake of the target sized microbubbles maintains not less than 80% of the target bubble concentration when maintained at a temperature not greater than 25° C. for a duration not less than 4 weeks from a time of containing the cake in the container.

As may be appreciated, in contrast to prior approaches, the presently described subject matter may utilize a relatively high volume fraction of microbubbles during the size isolation process. In one embodiment, the microbubble fraction in the solution may be not less than 20% and not greater than 30%.

In addition, control of the temperature of the solution may assist in a high yield process. For instance, the solution may be at a temperature of not less than 1° C. and not greater than 15° C. Alternatively, the solution is at a temperature of not greater than 4° C.

Furthermore, the viscosity of the resulting cake of the microbubble solutions may be controlled to enhance stability while maintaining characteristics favorable for use in medical procedures. For instance, the microbubble product resulting from the high yield size isolation process may be stored at room temperature or at a refrigerated temperature. In addition, the viscosity at these temperatures may be controlled to reduce the diffusion forces acting on the microbubbles in the size isolated microbubble product. As such, the cake of the target sized microbubbles may have a viscosity of at least about 0.0028 Pa·s at a temperature of 4° C. In addition, the cake of the target sized microbubbles may have a viscosity of at least about 0.0016 Pa·s at a temperature of 25° C. Furthermore, the resulting size isolated microbubble product may have favorable characteristics at body temperature (e.g., for certain medical contexts in which the microbubbles may be employed). In this regard, the cake of the target sized microbubbles may have a viscosity of at least about 0.0013 Pa·s at a temperature of 37° C.

A second aspect described herein includes a method for isolating target sized microbubbles from a polydisperse population of microbubbles at high yield, which includes controlling a plurality of diffusion factors for a suspension of a polydisperse population of microbubbles to maintain a target diffusion coefficient of the suspension at not less than about 1.0×10⁻¹⁸ mm²/s and not greater than about 1.0×10⁻¹³ mm²/s. The maintaining of the diffusion coefficient includes maintaining a temperature of the solution at not less than about 1° C. and not greater than about 15° C. Furthermore, the maintaining includes providing the population of microbubbles in the solution at a microbubble volume fraction of not less than 20% of the solution and not greater than 50% of the solution. In turn, the method includes applying a centrifugal field to the suspension of the polydisperse population of microbubbles having the target gas diffusion coefficient to isolate the target sized microbubbles from the solution.

A number of feature refinements and additional features are applicable to the second aspect. These feature refinements and additional features may be used individually or in any combination. As such, each of the following features that will be discussed may be, but are not required to be, used with any other feature or combination of features of the second aspect.

In an embodiment of the second aspect, the applying may include generating a monodisperse cake of the target sized microbubbles having a target microbubble concentration of not less than about 1.0×10⁹microbubbles(MB)/mL. The target sized of the target sized microbubbles may include a monodisperse microbubble diameter of not smaller than about 0.5 μm and not larger than about 10 μm. Alternatively, the target sized of the target sized microbubbles may include a monodisperse microbubble diameter of not smaller than about 4 μm and not larger than about 5 μm. In other embodiments, the monodisperse microbubble diameter of not smaller than about 1 μm and not larger than about 2 μm, not smaller than about 3 μm and not larger than about 4 μm, not smaller than about 4 μm and not larger than about 5 μm, or not smaller than about 5 μm and not larger than about 8 μm.

In an embodiment, the method may include containing the monodisperse cake of the target sized microbubbles at the target microbubble concentration in a sealed container. For instance, the sealed container may be a syringe or a vial. In any regard, the sealed container may have an internal volume and a volume of the monodisperse cake is substantially the same as the internal volume such that no fluid headspace is present in the sealed container containing the cake.

As described above, a resulting microbubble product of the high yield size isolation process may provide increased stability for the microbubbles. In an embodiment, the monodisperse cake of the target sized microbubbles may maintain not less than 90% of the target bubble concentration when maintained at a temperature not greater than 4° C. for a duration not less than 4 weeks from a time of the containing the cake in the sealed container. Alternatively, the monodisperse cake of the target sized microbubbles may maintain not less than 80% of the target bubble concentration when maintained at a temperature not greater than 25° C. for a duration not less than 4 weeks from a time of the containing the cake in the sealed container.

A third aspect of the disclosure includes a method for isolation of target sized microbubbles in a target size range from a composition of polydisperse microbubbles with high yield. The method includes producing a polydisperse population of microbubbles from a lipid suspension. The polydisperse population of microbubbles comprises target sized microbubbles of a target size in a target size range and undesired microbubbles of an undesired size outside of the target size range. The method also includes preparing a suspension of the polydisperse population of microbubbles at a target microbubble volume fraction in an aqueous media and controlling one or more gas diffusion factors of the suspension to achieve a target gas diffusion coefficient at least in part based on the target microbubble volume fraction and the target size range. In turn, the method includes applying a centrifugal field to the suspension to isolate the target sized microbubbles.

A number of feature refinements and additional features are applicable to the third aspect. These feature refinements and additional features may be used individually or in any combination. As such, each of the following features that will be discussed may be, but are not required to be, used with any other feature or combination of features of the third aspect.

For instance, in an embodiment the one or more gas diffusion factors may include a fluid viscosity of the suspension. As such, the method may also include adding a viscosity modifier to the second suspension to increase the fluid viscosity of the suspension to achieve the target gas diffusion coefficient. As an example, the viscosity modifier may be glycerol.

Furthermore, the fluid viscosity of the suspension may be an effective fluid viscosity based on the target microbubble volume fraction of microbubbles in the suspension. In this regard the target microbubble volume fraction of microbubbles in the second suspension may be greater than 20% and not greater than 50%. Furthermore, the target size range for the resulting size isolated microbubble product may be from not less than about 0.5 μm and not greater than about 10 μm. In another embodiment, the target size range may be from not less than about 4 μm and not greater than about 5 μm. In other embodiments, the target size for the microbubble diameter may be not smaller than about 1 μm and not larger than about 2 μm, not smaller than about 3 μm and not larger than about 4 μm, not smaller than about 4 μm and not larger than about 5 μm, or not smaller than about 5 μm and not larger than about 8 μm.

In an embodiment, the one or more gas diffusion factors may include a temperature of the suspension. The temperature of the suspension may not be greater than about 4° C.

A fourth aspect of the present disclosure includes a pharmaceutical product comprising a stable pharmaceutical monodisperse microbubble composition. The composition may include an aqueous media and a concentrated population of monodisperse microbubbles in a target microbubble size range and at a target microbubble concentration in the aqueous media of not less than about 1.0×10⁹ MB/mL. The aqueous media and the concentrated population of monodisperse microbubbles comprise the stable pharmaceutical monodisperse microbubble composition having a composition volume. The composition may also include a sealed container having an internal container volume in which the stable pharmaceutical monodisperse microbubble composition is disposed The composition volume is substantially equal to the internal container volume.

A number of feature refinements and additional features are applicable to the fourth aspect. These feature refinements and additional features may be used individually or in any combination. As such, each of the following features that will be discussed may be, but are not required to be, used with any other feature or combination of features of the fourth aspect. For instance, the target microbubble concentration in the aqueous media may be obtained by controlling a gas diffusion coefficient of a suspension comprising target sized microbubbles during centrifugal isolation of the target sized microbubbles. Furthermore, a microbubble volume fraction of the stable pharmaceutical monodisperse microbubble composition is at least about 70%.

In an embodiment, the stable pharmaceutical monodisperse microbubble composition may be in an injectable form and the sealed container comprises an injection device. Specifically, the injection device may be a syringe. In other embodiments, the container may be a vial

As described above, it may be advantageous to provide increased viscosity for storage of the composition while maintaining an identified viscosity at body temperature for some applications. As such, the stable pharmaceutical monodisperse microbubble composition may have a viscosity of at least about 0.0028 Pa·s at a temperature of 4° C., may have a viscosity of at least about 0.0016 Pa·s at a temperature of 25° C., and may have a viscosity of at least about 0.0013 Pa·s at a temperature of 37° C.

Various aspects and example implementation embodiments of the methods and systems of the disclosure are presented in the following description, including the claims, and in the drawings.

### BRIEF DESCRIPTION OF THE DRAWINGS

FIG. 1 depicts an embodiment of a method for microbubble production with control of parameters regarding a diffusion force acting on the microbubbles in the solution undergoing size isolation.

FIG. 2 depicts an embodiment of a system for production of a polydisperse solution of microbubbles.

FIG. 3 depicts an embodiment of a separation column in which a solution comprising a polydisperse solution microbubbles may be disposed during the size isolation process.

FIG. 4 depicts a detailed view of various forces acting on the microbubbles in a separation column during centrifugation for size isolation microbubbles.

FIG. 5 depicts an embodiment of a method for control of the number of diffusion parameters for increased yield during size isolation microbubbles.

FIG. 6 depicts a particular stage during the size isolation process for isolation of microbubbles of a target size.

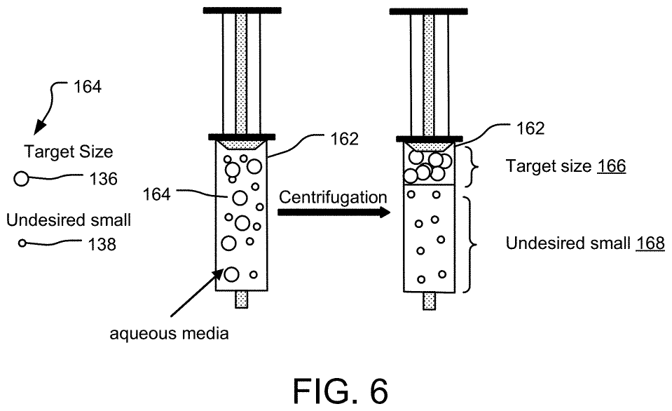

### DETAILED DESCRIPTION

While the invention is susceptible to various modifications and alternative forms, specific embodiments thereof have been shown by way of example in the drawings and are herein described in detail. It should be understood, however, that it is not intended to limit the invention to the particular form disclosed, but rather, the invention is to cover all modifications, equivalents, and alternatives falling within the scope of the invention as defined by the claims.

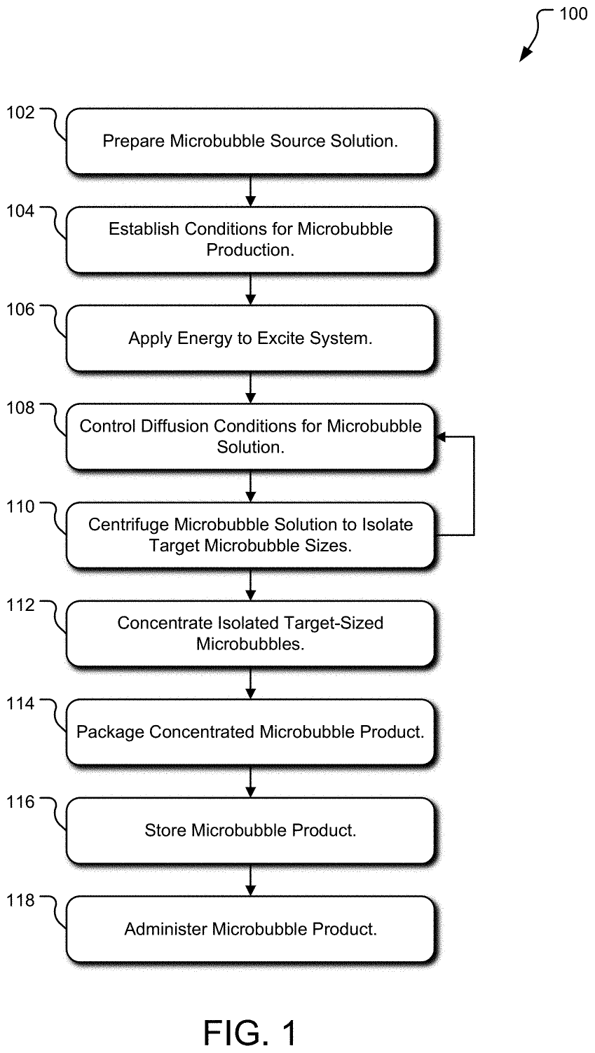

FIG. 1 includes a flow charts that depicts an embodiment of a method 100 for production of size isolated microbubbles at high-yield to produce a microbubble product. The method 100 may include preparing 102 a microbubble source solution. The source solution may be a combination of components from which microbubbles are initially formed to produce a polydisperse population of microbubbles. In an embodiment, this may include a lipid suspension for use in preparing lipid shelled microbubbles. The preparing 102 may include preparing a solution comprising a mixture of lipid phosphatidylcholine (PC) and a lipid with a polyethylene glycol (PEG) group. The ratio of PC to PEG may be at a 9:1 molar ratio. While any appropriate lipid may be utilized, the PC lipid may range from a chain length of 14-24. In other embodiments, headgroup modified lipids containing chemically bound groups, such as groups bound by covalent bonding or streptavidin-avidin linkage, may also be used in the solution. The PEG molecular weight may range from 2000-5000. The lipid components may be combined in an aqueous medium. In an embodiment, the aqueous medium may be phosphate buffered saline (PBS). In any regard, the lipid component may be mixed with the aqueous medium to provide a lipid solution. Other components may be added such as surfactants (e.g., polymeric surfactants) or proteins (e.g., albumin), which may provide more stable microbubbles. Further stabilizing agents or stabilizers may be added to the lipid solution as well. For instance, one or more stabilizers including a fatty acid, dextrose, medical grade honey, lecithin, pectin, xanthan gum, cholesterol, casein, gum Arabic, and/or fatty alcohols may be added, which may promote stability of resulting microbubbles as described in U.S. Provisional App. No. 62/791,423 filed on Jan. 11, 2019, the entirety of which is incorporated herein in its entirety.

The method 100 may include establishing 104 conditions for microbubble generation. This may include heating the lipid solution to above the phase transition temperature of the lipid solution to promote mixing of the lipid components in the aqueous medium. By heating the lipid solution to above the phase transition temperature, the lipid solution may be more solvent relative to the aqueous medium to promote mixing. Furthermore, the mixture may also be physically mixed while heated. The lipid solution may be further excite to break up large lipid aggregates into smaller micelles and liposomes. This may include applying energy using an energy applicator such as a sonicator or the like. The energy applicator may be the same as an energy applicator discussed below for excitation to produce microbubbles. However, the energy applicator may be operated at a lower power setting to promote mixing of the lipid component in the aqueous medium prior to generation of microbubbles. In any regard, the suspension may be energized (e.g., sonicated, agitated, or otherwise physically excited) until the solution is translucent and there are no visible aggregates. Once the mixture is translucent and no visible aggregates are present, the lipid solution may be cooled to below the phase transition temperature of the lipid component of the lipid solution. For instance, the temperature of the lipid solution may be set to no less than 5° C. below the phase transition temperature of the main phospholipid in the solution.

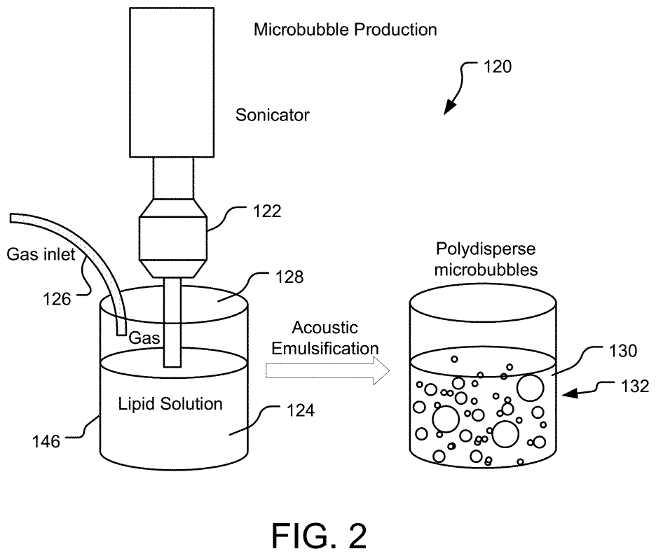

The establishing 104 of the conditions for microbubble production may also include introduction of a gas phase into a headspace of a vessel containing the lipid solution. With further reference to FIG. 2, an embodiment of a system 120 that may be utilized in relation to microbubble production is depicted. In this regard, the lipid solution 124 as described above may be provided according to the foregoing. The lipid solution 124 may be disposed in a vessel 146. In turn, a gas inlet 126 may be introduced into a headspace 128 of the vessel 146. In turn, a gas may be introduced into the headspace 128 at a pressure greater than atmospheric. Initially, the gas may be used to flush out or purge ambient air in the headspace 128. Thereafter, the headspace 128 may be maintained at an elevated pressure (e.g., to provide a slight indentation on the surface of the lipid suspension 124). The gas introduced in the headspace 120 may be any appropriate gas, but at least in one embodiment the gas may be perfluorobutane or other high molecular weight gas.

The method 100 may further include applying 106 energy to excite the system and create microbubbles. As describe above, this may include physical agitation of the lipid solution 124. With returned reference to FIG. 2, a sonicator 122 may be introduced such that the sonicator 122 is disposed at the interface between the pressurized gas in the headspace 128 and the lipid solution 124 in the vessel 146. In turn, the sonicator 122 may be activated to sonicate the interface and create the microbubbles through mechanical agitation (e.g., acoustic emulsification) on of the interface between the high pressure gas and the lipid solution 124. While a sonicator 122 is shown and discussed, other mechanisms for physical agitation of the interface between the high pressure gas and the lipid solution 124 may be utilized without limitation such as a mechanical agitator for shaking the lipid solution 124, a dental amalgamator for acting on the lipid solution 124, or an in-line homogenizer/colloid mill for acting on the lipid solution 124.

It is been found that the initial microbubble yield may be increased by exciting a relatively warm lipid solution 124. In this regard, the warm lipid solution may be maintained below the main lipid phase transition temperature of the solution, but maintained relatively close to the phase transition temperature (e.g., within 5° C. of the main lipid phase transition temperature of the solution). In this regard, the lipid solution 124 may be disposed relative to a thermal regulation device such as a heater and/or cooler to maintain the temperature of the lipid solution 124.

Once initial microbubble production has been completed, the method 100 may include controlling 108 one or more diffusion parameters to maintain preferable diffusion conditions for the microbubble suspension 132. The microbubble suspension 132 may be maintained in the aqueous solution of the lipid solution 124 or may be collected and transferred to a virgin aqueous solution. Further still, the microbubble suspension 132 may be centrifuged to collect the polydisperse microbubble population in an supernatant cake for collection. In any regard, further details regarding the controlling 108 of the diffusion parameters is described in greater detail below. However, the controlling 108 may include establishing conditions in which the diffusion forces acting on the microbubbles in a solution are maintained within a predetermined range to reduce or minimize the diffusion forces in the microbubble suspension 132. Specifically, it is noted that to provide increased yield of lipid stabilized, size isolated microbubbles using differential centrifugation, it is important to optimize initial bubble production while minimizing factors the results in microbubble gas dissolution and breakdown of microbubbles during centrifugation wash cycles of the size isolation process. In this regard, it is presently recognized that it is possible to reduce or impede gas dissolution of the microbubble by minimizing the diffusion force that governs bubble the solution. Specifically, the Stokes-Einstein's Brownian diffusion equation for gas dissolution provides characterization of the gas dissolution of a system comprising microbubbles with a diffusion constant D. The Stokes-Einstein's Brownian diffusion equation can be represented as:

$$D = \frac{kT}{6 π {μ}^{*} r} \qquad Equation 1$$

where k is the gas constant, T as the temperature of the ambient fluid, μ* as the effective viscosity of the fluid, and r in the radius of the microbubble. In addition, it is recognized that the effective viscosity of the fluid medium is at least in part based upon the viscosity of the fluid containing the microbubbles and the volume fraction of the microbubbles in the fluid. Specifically, a model to determine the effective fluid viscosity in view of crowding and bubble interaction is provided as:

$$\frac{{μ}^{*}}{μ} = 1 + 2.5 Φ + 7.6 {Φ}^{2} \qquad Equation 2$$

where μ* is the effective viscosity of the solution, μ is the viscosity of the fluid in which the microbubbles are provided, and Φ as the bubble volume fraction. As will be described in greater detail below, any of the parameters capable of being controlled in relation to the system from Equation 1 may be controlled 108 to reduce or minimize the diffusion forces acting on the microbubbles in the microbubble solution 132.

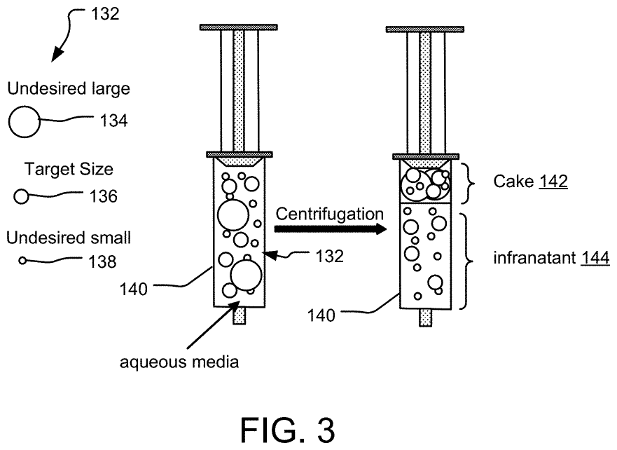

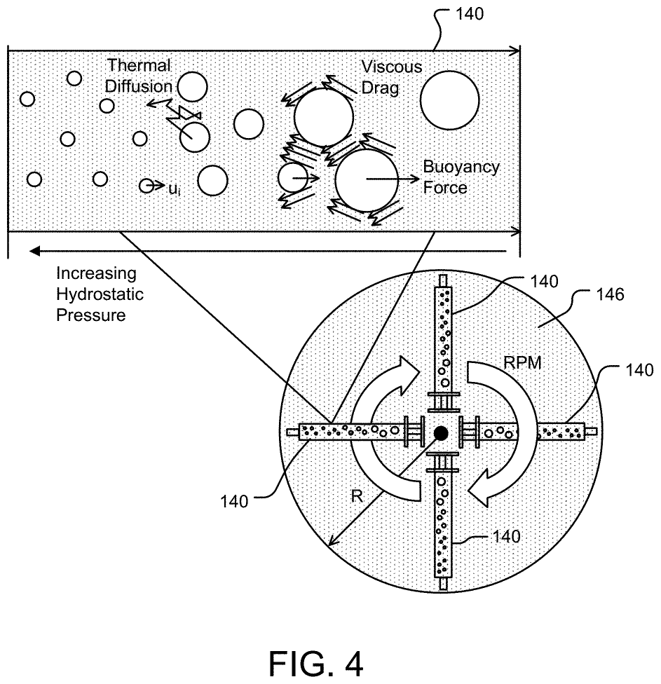

In any regard, the method 100 may include centrifuging 110 the microbubble solution to isolated target microbubble sizes. This may include a multiple step centrifugal wash cycle in which the solution may be subjected to a number of centrifuging washes to isolate a given target size microbubble. With reference to FIG. 3, one example of a wash cycle is illustrated. As can be appreciated, a separation column 140 may contain a microbubble solution 132 that includes undesired large microbubbles 134, target sized microbubbles 136, and undesired small microbubbles 138. With additional reference to FIG. 4, a detailed view of a separation column 140 in a centrifuge 146 is shown. As can be appreciated, upon application of a relative centrifugal force (RCF) to the separation column 140, differential centrifugation may occur to effectively separate the polydisperse population of microbubbles based on size in view of the forces acting on the microbubbles as shown in FIG. 4.

The separation column 140 may be subjected to centrifugation to produce a supernatant cake or simply cake 142 and an infranatant 144. In this regard, a number of wash cycles may be applied to selectively isolate various sized microbubbles in the cake 142 or infranatant 144, depending on the wash cycle. For example, the centrifuging 110 may include applying a first centrifugal field having a first field strength to a suspension comprising a polydisperse population of microbubbles for a first duration of time, thereby forming a first infranatant 144 comprising at least a portion of target microbubbles and a first cake 142 comprising microbubbles having a greater size than the target microbubbles. In turn, the first cake 142 may be removed. In turn, the first infranatant 144 may be have applied thereto a second centrifugal field having a second field strength to the first infranatant for a second duration of time, the second field strength to form a second supernatant cake 142 comprising at least a portion of the target microbubbles and second infranatant 144 comprising microbubbles having a smaller size than the target microbubbles. Thus, the second supernatant cake 142 that contains the target size microbubbles may be isolated.

In this regard, it may be appreciated that because the diffusion coefficient that describes the diffusion forces acting on a microbubble in the microbubble solution 132 may be at least in part based on the size (e.g., radius) of the microbubble, the controlling 108 of the diffusion factors may be based on the target size of the microbubble targeted in the centrifuging 110. In any regard, once isolated, the target size microbubbles may be concentrated 112.

As will be discussed in greater detail below, by facilitating high-yield size isolated microbubble populations, a relatively large volume of highly concentrated size isolated microbubbles may be realized. In this regard, the resulting size isolated microbubble product may provide very high volume fractions in a resultant solution. As can be appreciated from the foregoing equations, such a high volume fraction may result in a high effective viscosity of a final size isolated microbubble product. This may result in a low diffusion coefficient, indicative of low diffusion forces in the concentrated size isolated microbubble product. Accordingly, the resulting high-yield, size isolated microbubble product may demonstrate improved stability as the diffusion forces associated with microbubble gas dissolution and breakdown may be minimized.

As such, the resulting microbubble product may be packaged 114 in an appropriate form. Any suitable vessel may be utilized for containment of the packaged 114 concentrated microbubble product. However, certain product forms are specifically contemplated herein such as a vial or syringe to contain the concentrated microbubble product. Advantageously, when contained in a syringe, the resulting concentrated microbubble product may be injectable without further handling of the microbubbles. By minimizing the handling or transfer of the size isolated microbubbles, the high-yields of the foregoing process may be maintained with minimal degradation of the microbubbles due to transfer between vessels, which may result in breakdown or other degradation of the microbubbles. In an embodiment, the internal volume of the vessel into which the concentrated microbubble product is provided may be the same as the volume of the concentrated microbubble product itself. That is, the container into which the concentrated microbubble product is provided may have substantially no headspace such that the concentrated microbubble product occupies substantially all of the internal volume of the vessel. This may also assist in reduction of the dissolution forces that tend to break down microbubbles during storage.

As such, the improved stability of the concentrated microbubble product may allow for storage 116 of the microbubble product. The storage 116 may be at room temperature or may be at a refrigerated temperature to promote microbubble preservation. For instance, when stored at a refrigerated temperature (e.g., not greater than 4° C.), at least 90% of the initial volume of the concentrated microbubble product disposed in the containment vessel during the packaging 114 may remain after not less than 4 weeks of storage 116. In addition, when stored at room temperature (e.g., not greater than 23° C.), at least 80% of the initial volume of the concentrated microbubble product disposed in the containment vessel during the packaging 114 may remain after not less than 4 weeks storage. As may be appreciated, the increased stability of the microbubble product may allow for enhanced shelf life, thus facilitating maintaining the microbubble product in inventory at a medical facility prior to use. This may allow for increased flexibility in relation to the use of the microbubble product for various medical applications such as use as a contrast agent in medical imaging, ultrasound targeted drug delivery, ultrasound targeted ablation of tissue, ultrasound aided opening of tissue or any other medical application in which size isolated microbubbles may be utilized. Additionally or alternatively, the microbubble product may be used in conjunction with ultrasound imaging as noninvasive and targeted method to increase biomarker availability for liquid biopsies in the early detection of various diseases. The microbubble product may also be used to predict pre-symptomatic diabetes with ultrasound imaging (e.g., involving ultrasound imaging of the pancreas islets). In still another context, the microbubble product may be used with sonoporation with ultrasound to increase available biomarkers in the blood for liquid biopsy and early disease detection. Thus, the method 100 may further include administering 118 the microbubble product and a medical application.

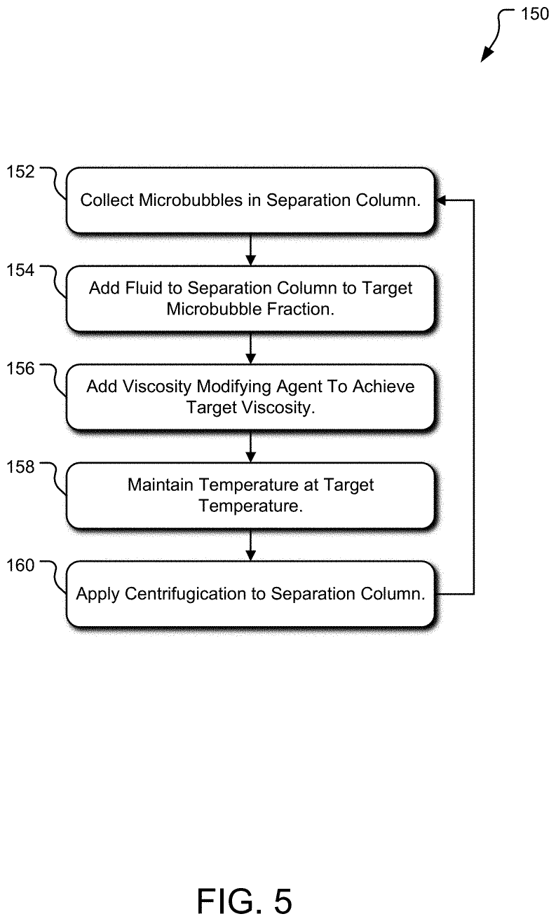

With further reference to FIG. 5, a method 150 for controlling one or more diffusion factors for a microbubble solution 132 during application of centrifugation for size isolation is shown. The method 150 may include collecting 152 microbubbles into separation column 140. As shown in FIG. 3, the separation column may be a syringe, which may also be used to store 116 a resulting size isolated microbubble product. The method 150 may further include adding 154 fluid to the separation column to achieve a target microbubble fraction. As discussed above, increases in the target microbubble fraction may result in reduction in the diffusion coefficient, and thus the diffusion forces acting on the microbubbles in the separation column during application of centrifugation for size isolation. In an embodiment, the target microbubble fraction may be not less than about 20% and not greater than about 50%. However, in other approaches the target microbubble fraction may be not less than 25%, not less than 30%, or not less than 40%. Further still, the target microbubble fraction may be not greater than about 45%, not greater than about 40%, or not greater than about 30%. Furthermore, various target microbubble fraction ranges may be provided according to any of the minimum and maximum values listed above, provided that the maximum value is greater than the minimum value of the range. In this regard, by maintaining a microbubble fraction at least about 20%, the effective viscosity of the solution may be increased, thus decreasing the diffusion forces per Equation 1 above.

In addition, while microbubble fraction is recognized as affecting the effective viscosity of the solution, the viscosity of the fluid in which the microbubbles are contained is also a factor effective viscosity. In this regard, the viscosity of the fluid in which the microbubbles are provided may also be controlled (e.g., to increase the viscosity of the solution and thus reduce the diffusion coefficient of the system). In this regard, a viscosity modifier may be added 156 to the microbubble solution to target effective viscosity. In an embodiment, the viscosity modifier may comprise glycerol

Furthermore, the temperature of the solution may also affect the diffusion coefficient governing the diffusion forces acting on the microbubbles in the solution. As such, the method 150 may include maintaining 158 a temperature of the solution at a target temperature. As described above, during initial microbubble production, the lipid solution from which the microbubbles are produced may be preferably maintained at below about 5° C. less than the phase transition temperature of the lipid solution. In this regard, the solution during initial microbubble production may be at a relatively elevated temperature. As such, the maintaining 158 of the temperature of the solution may include reducing the temperature of the solution to well below that of the temperature at which the microbubbles are initially produced from the lipid solution. This may include extraction of the microbubbles from the lipid solution remaining after initial bubble production. In this regard, the extracted microbubbles may be refrigerated or may be introduced into a fluid (e.g., during adding 154 of the fluid to the separation column) that is at a refrigerated temperature. For instance, the target temperature of the solution during centrifugation for size isolation may be not greater than about 1° C., not greater than about 4° C., not greater than about 5° C., not greater than about 10° C., or not greater than about 15° C. The method 150 may also include applying 160 centrifugation to the separation column for size isolation of a target size microbubble. The applying 160 may be performed in view of the prior steps of the method 150 that effectively control the diffusion parameters of the system. As may be appreciated in FIG. 5, the method 150 may iterate such that after application 160 of centrifugation to the separation column, the method 150 may repeat. In this regard, and each successive iteration of the method 150, the control of the various diffusion parameters provided in the steps of the method 150 may be altered based upon the particular centrifugal washing being applied. For instance, if different sized microbubbles are targeted and subsequent washes, the diffusion parameters maintained may vary.

The description of a feature or features in a particular combination do not exclude the inclusion of an additional feature or features in a variation of the particular combination. Processing steps and sequencing are for illustration only, and such illustrations do not exclude inclusion of other steps or other sequencing of steps to an extent not necessarily incompatible. Additional steps may be included between any illustrated processing steps or before or after any illustrated processing step to an extent not necessarily incompatible.

The terms “comprising”, “containing”, “including” and “having”, and grammatical variations of those terms, are intended to be inclusive and nonlimiting in that the use of such terms indicates the presence of a stated condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or any appropriate grammatical variation of such narrower terms). For example, a statement that something “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. As used herein, a range for a feature refers to one or more values for that feature within an upper limit and lower limit, inclusive of the upper and lower limits, and includes situations in which the upper limit and the lower limit are the same, that is when the range includes a single value represented by the equal upper and lower limits.

### EXAMPLES

### Process Summary

Polydisperse microbubbles are prepared by excitation methods such as acoustic emulsification. In the first stage of size-isolation, bubbles below an undesired size are extracted from the polydisperse suspension of high volume fraction (20-30%) using a large capacity column. Since, the bubble population extracted will be at a lower concentration (and lower volume fraction) than the initial population, subsequent separation to remove the undesired smaller sizes is performed by adjusting bubble volume fraction and or fluid viscosity in the next separation column. All centrifugal washing is done with cold aqueous media to control temperature.

### Preparation Of The Lipid Suspension

1. 1 to 5 mg/mL lipid suspension is prepared by weighing a mixture of main lipid phosphatidylcholine (PC) and lipid containing a polyethylene glycol (PEG) group at a 9:1 molar ratio. The PC lipid can range from a chain length of 14-24. The PEG molecular weight can range from 2000-5000.
2. The mixture is heated (under mixing) to above the phase transition temperature of the PC lipid.
3. The lipid mixture is further sonicated to break up large lipid aggregates into smaller micelles and liposomes by immersing the tip of a Branson Sonifier® 250/450 probe sonicator into the middle of the suspension and applying half the power output until the solution is translucent and there are no visible aggregates.
4. The lipid mixture is set to at least 5° C. below the phase transition temperature of the main phospholipid.

### Polydisperse Microbubble Production at High Yield

1. The gas phase (perflourobutane or high molecular weight gas) is introduced to the headspace at an overpressure that flushes out ambient air and causes a greater than about 0.5 inch indentation on the surface of the suspension.
2. The microbubbles are prepared by acoustic emulsification (probe sonication) by sonicating a full power at the interface of the gas and the warm lipid suspension. Results demonstrate that the initial bubble yield increases dramatically when a warm lipid solution (below the main lipid phase transition temperature) is sonicated during production as opposed to a cold one.
3. Microbubbles are carefully collected and drawn into large columns (30 mL or 60 mL) with care taken not to collect undesirable the macroscopic bubble foam residue.
4. The syringe columns are centrifuged at higher field force to collect all microbubbles in cake (top phase) and non-gas microbubble particles such as liposomes and micelles into infranatant (bottom phase). For example, 300 RCF for 5 minutes is used in 30 mL column to collect all microbubbles. The concentrated polydisperse microbubbles are collected and infranatant is discarded or recycled.

### Stage 1: Extracting Target Sizes And Small Sizes From Undesired Large Sizes Using Column At High Bubble Fraction And Cold Media:

1. The microbubbles cakes produced are combined into a separation column (30 ml or 60 mL) by diluting the cakes and combining them into a separation column using cold aqueous media (i.e. pH 7.4 saline). The resulting suspension is diluted to a volume fraction of 20-30%.
2. Lower strength field is applied to cause all bubbles of undesired large size and above to collect into the cake.
3. The infranatant containing few or substantially none of the undesired large sizes and mixture of desired size target and undesired small are collected, concentrated and then transferred to another separation column via syringe to syringe transfer.

### Stage 2: Removing Undesired Small Sizes From Target Sizes While Maintaining High Bubble Fraction And Washing With Cold Media:

1. The column contents are carefully adjusted to 20-30% volume fraction of microbubbles with cold aqueous media in order to maintain the high volume fraction/high fluid viscosity and low temperature.
2. High field centrifugal force is applied to cause the target size to form into the cake and the undesired small sizes to remain in the infranatant.
3. Due to the nature of greater abundance of the smaller undesirable sizes, the cake is reconstituted with the cold aqueous media and the high field centrifugal force is applied in multiple cycles to remove undesired small sizes from the target size.
4. The final target size is concentrated and transferred to storage container.

This method may allow for the production of four different size classes at high yield including 2 micron bubbles at a yield of greater than 2×10¹⁰ MB/mL, 3 micron bubbles at a yield of 1×10¹⁰ MB/mL, 4 micron bubbles at a yield of 8×10⁹ MB/mL and 6 micron bubbles at a yield of 2×10⁹ MB/mL.

### Effect Of Bubble Fraction And Temperature On Yield Of Size-Isolated Microbubbles

a. Maintaining High Bubble Fraction Versus Reducing Bubble Fraction—Cold Temperature Washing

The table below provides a comparison of the yield of size sorted bubbles for maintaining a high bubble (25%) fraction/high viscosity versus reducing the bubble fraction from high (25%) to low (6%) in the washing stages. The same bubble production method is used for both test columns and cold aqueous media is adopted for centrifugal washing. The cake yield for maintaining a high bubble fraction (25%) is approximately 4 fold greater than reducing bubble fraction to 6%. The microbubble yield for maintaining a high bubble (25%) fraction is approximately 10 fold greater than reducing bubble fraction to 6%.

Comparison Of Size-Isolation By Maintaining High Bubble
Fraction Vs Lowering Bubble Fraction At Same T

| | High (25%) to high (25%) | High (25%) to low (6%) |
|---|---|---|
| | bubble fraction | bubble fraction |
| Cake Yield | 2 mL | 0.5 mL |
| Microbubble | 1e10 microbubbles | 1e9 microbubbles |
| yield | | |

b. Cold (5° C.) Vs Room Temperature (22° C.) Aqueous Media At High Bubble Fraction

The table below provides a comparison of the yield of size sorted bubbles when adopting aqueous media at temperature of 5° C. versus 22° C. and maintaining a high bubble fraction (25%) during differential centrifugation. The cake yield for 5° C. is ˜2 fold greater than 22° C. The microbubble yield for 5° C. is approximately 3 fold greater than 22° C.

Comparison of Size-Isolation using Cold vs Warm
media while maintaining high bubble fraction

| | 5° C. media | 22° C. media |
|---|---|---|
| Cake Yield | 2 mL | 1 mL |
| Microbubble yield | 1e10 microbubbles | 3.0e9 microbubbles |

### Example Values for Diffusion Coefficients for High Yield Processing

The following represents example diffusion coefficients for a number of potential conditions for high yield microbubble isolation. Throughout the following, the following, a Boltzmann's constant of k=1.38E-23 J/K is presumed.

In one example, water is used as a fluid medium in which the microbubbles are provided. As such, viscosity values at various temperatures are provided as follows:

μ(water) at 1° C.=0.00173 Pa·s

μ(water) at 25° C.=0.00089 Pa·s

As such, diffusion coefficients based on microbubble diameter for a bubble fraction of Φ=5% are provided when the effective viscosity is μ*(5%, 1° C.)=0.00197912 Pa·s, the values being:

| Diameter (μm) | Diffusion Constant (m²/s) |
|---|---|
| 2 | 1.01E−13 |
| 4 | 5.07E−14 |
| 6 | 3.38E−14 |
| 8 | 2.54E−14 |

With water at an elevated temperature of 25° C., the effective viscosity is Φ=25° C.)=0.00101816 Pa·s, and the diffusion equation values based on target microbubble size are:

| Diameter (μm) | Diffusion Constant (m²/s) |
|---|---|
| 2 | 2.14E−13 |
| 4 | 1.07E−13 |
| 6 | 7.15E−14 |
| 8 | 5.36E−14 |

Diffusion coefficients based on microbubble diameter for a bubble fraction of c=25% are provided such that the effective viscosity is μ*(25% , 1° C.)=0.003633 Pa·s, the values being:

| Diameter (μm) | Diffusion Constant (m²/s) |
|---|---|
| 2 | 5.52E−14 |
| 4 | 2.76E−14 |
| 6 | 1.84E−14 |
| 8 | 1.38E−14 |

With water at an elevated temperature of 25° C., the effective viscosity is μ*(25% , 25° C.)=0.001869 Pa·s, and the diffusion constant values based on target microbubble size are:

| Diameter (μm) | Diffusion Constant (m²/s) |
|---|---|
| 2 | 1.17E−13 |
| 4 | 5.84E−14 |
| 6 | 3.89E−14 |
| 8 | 2.92E−14 |

Diffusion coefficients based on microbubble diameter for a bubble fraction of Φ=50% are provided such that the effective viscosity is μ*(50% , 1° C.)=0.0071795 Pa·s, the values being:

| Diameter (μm) | Diffusion Constant (m²/s) |
|---|---|
| 2 | 2.80E−14 |
| 4 | 1.40E−14 |
| 6 | 9.32E−15 |
| 8 | 6.99E−15 |

With water at an elevated temperature of 25° C., the effective viscosity is μ*(50%, 25° C.)=0.0036935 Pa·s, and the diffusion constant values based on microbubble size are:

| Diameter (μm) | Diffusion Constant (m²/s) |
|---|---|
| 2 | 5.91E−14 |
| 4 | 2.95E−14 |
| 6 | 1.97E−14 |
| 8 | 1.48E−14 |

Alternatively, glycerol may be used as a fluid medium in which the microbubbles are provided. As such, viscosity values at various temperatures are provided as follows:

μ(glycerol) at 1° C.=10.693 Pa·s

μ(glycerol) at 25° C.=0.905 Pa·s

Diffusion coefficients based on microbubble diameter for a bubble fraction of Φ=5% are provided such that the effective viscosity is μ*(5%, 1° C.)=12.232792 Pa·s, the values being:

| Diameter (μm) | Diffusion Constant (m²/s) |
|---|---|
| 2 | 1.64E−17 |
| 4 | 8.20E−18 |
| 6 | 5.47E−18 |
| 8 | 4.10E−18 |

With glycerol at an elevated temperature of 25° C., the effective viscosity is μ*(5%, 25° C.)=1.03532 Pa·s, and the diffusion constant values based on target microbubble size are:

| Diameter (μm) | Diffusion Constant (m²/s) |
|---|---|
| 2 | 2.11E−16 |
| 4 | 1.05E−16 |
| 6 | 7.03E−17 |
| 8 | 5.27E−17 |

Diffusion coefficients based on microbubble diameter for a bubble fraction of c=25% are provided such that the effective viscosity is μ*(25% , 1° C.)=22.4553 Pa·s, the values being:

| Diameter (μm) | Diffusion Constant (m²/s) |
|---|---|
| 2 | 8.94E−18 |
| 4 | 4.47E−18 |
| 6 | 2.98E−18 |
| 8 | 2.23E−18 |

With glycerol at an elevated temperature of 25° C., the effective viscosity is μ*(25%, 25° C.)=1.9005 Pa·s, and the diffusion constant values based on target microbubble size are:

| Diameter (μm) | Diffusion Constant (m²/s) |
|---|---|
| 2 | 1.15E−16 |
| 4 | 5.74E−17 |
| 6 | 3.83E−17 |
| 8 | 2.87E−17 |

Diffusion coefficients based on microbubble diameter for a bubble fraction of Φ=50% are provided such that the effective viscosity is μ*(50% , 1° C.)=44.37595 Pa·s, the values being:

| Diameter (μm) | Diffusion Constant (m²/s) |
|---|---|
| 2 | 4.52E−18 |
| 4 | 2.26E−18 |
| 6 | 1.51E−18 |
| 8 | 1.13E−18 |

With glycerol at an elevated temperature of 25° C., the effective viscosity is μ*(50%, 25° C.)=3.75575 Pa·s, and the diffusion constant values based on target microbubble size are:

| Diameter (μm) | Diffusion Constant (m²/s) |
|---|---|
| 2 | 5.81E−17 |
| 4 | 2.91E−17 |
| 6 | 1.94E−17 |
| 8 | 1.45E−17 |

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that only the preferred embodiment and variants thereof have been shown and described and that all changes and modifications that come within the spirit of the invention are desired to be protected.

1. A method for isolation of target sized microbubbles from a polydisperse population of microbubbles at high yield, the method comprising:
preparing a solution comprising the polydisperse population of microbubbles;
maintaining a microbubble volume fraction in the solution at greater than about 20% and not greater than about 50%; and
applying a centrifugal field to the solution to isolate the target sized microbubbles from the solution to produce a cake of the target sized microbubbles at a target microbubble concentration of not less than about 1.0×10⁹ microbubbles(MB)/mL.

2. The method of claim 1, further comprising:
containing the cake of the target sized microbubbles at the target microbubble concentration in a sealed container.

3.-4. (canceled)

5. The method of claim 2, wherein the sealed container comprises an internal volume and a volume of the cake is substantially the same as the internal volume such that no fluid headspace is present in the sealed container containing the cake.

6. The method of claim 5, wherein the cake of the target sized microbubbles maintains not less than 90% of the target bubble concentration when maintained at a temperature not greater than 4° C. for a duration not less than 4 weeks from a time of containing of the cake in the container.

7. (canceled)

8. The method of claim 1, wherein the microbubble fraction in the solution is not less than 20% and not greater than 30%.

9. The method of claim 8, wherein the solution is at a temperature of not less than 1° C. and not greater than 15° C.

10. The method of claim 8, wherein the solution is at a temperature of not greater than 4° C.

11. The method of claim 1, wherein the cake of the target sized microbubbles has a viscosity of at least about 0.0028 Pa·s at a temperature of 4° C.

12. The method of claim 11, wherein the cake of the target sized microbubbles has a viscosity of at least about 0.0016 Pa·s at a temperature of 25° C.

13. The method of claim 12, wherein the cake of the target sized microbubbles has a viscosity of at least about 0.0013 Pa·s at a temperature of 37° C.

14. A method for isolating target sized microbubbles from a polydisperse population of microbubbles at high yield, the method comprising:
controlling a plurality of gas diffusion factors for a suspension of a polydisperse population of microbubbles to maintain a target gas diffusion coefficient of the suspension at not less than about 1.0×10⁻¹⁸ mm²/s and not greater than about 1.0×10⁻¹³ mm²/s at least by:
maintaining a temperature of the solution at not less than about 1° C. and not greater than about 15° C., and
providing the population of microbubbles in the solution at a microbubble volume fraction of not less than 20% of the solution and not greater than 50% of the solution; and
applying a centrifugal field to the suspension of the polydisperse population of microbubbles having the target gas diffusion coefficient to isolate the target sized microbubbles from the solution.

15. The method of claim 14, wherein the applying comprises generating a monodisperse cake of the target sized microbubbles having a target microbubble concentration of not less than about 1.0×10⁹ microbubbles(MB)/mL.

16. The method of claim 14, wherein a target sized of the target sized microbubbles comprises a microbubble diameter of not smaller than about 0.5 μm and not larger than about 10 μm.

17. The method of claim 14, wherein a target sized of the target sized microbubbles comprises a microbubble diameter of not smaller than about 4 μm and not larger than about 5 μm.

18. The method of claim 14, further comprising:
containing the monodisperse cake of the target sized microbubbles at the target microbubble concentration in a sealed container.

19.-20. (canceled)

21. The method of claim 18, wherein the sealed container comprises an internal volume and a volume of the monodisperse cake is substantially the same as the internal volume such that no fluid headspace is present in the sealed container containing the cake.

22. The method of claim 21, wherein the monodisperse cake of the target sized microbubbles maintains not less than 90% of the target bubble concentration when maintained at a temperature not greater than 4° C. for a duration not less than 4 weeks from a time of the containing the cake in the sealed container.

23. The method of claim 21, wherein the monodisperse cake of the target sized microbubbles maintains not less than 80% of the target bubble concentration when maintained at a temperature not greater than 25° C. for a duration not less than 4 weeks from a time of the containing the cake in the sealed container.

24. A method for isolation of target sized microbubbles in a target size range from a composition of polydisperse microbubbles with high yield, comprising:
producing a polydisperse population of microbubbles from a lipid suspension, wherein the polydisperse population of microbubbles comprises target sized microbubbles of a target size in a target size range and undesired microbubbles of an undesired size outside of the target size range;
preparing a suspension of the polydisperse population of microbubbles at a target microbubble volume fraction in an aqueous media;
controlling one or more gas diffusion factors of the suspension to achieve a target gas diffusion coefficient at least in part based on the target microbubble volume fraction and the target size range; and
applying a centrifugal field to the suspension to isolate the target sized microbubbles.

25.-33. (canceled)

34. A pharmaceutical product comprising a stable pharmaceutical monodisperse microbubble composition, the pharmaceutical product comprising:
an aqueous media;
a concentrated population of monodisperse microbubbles in a target microbubble size range and at a target microbubble concentration in the aqueous media of not less than about 1.0×10⁹ MB/mL, wherein the aqueous media and the concentrated population of monodisperse microbubbles comprise the stable pharmaceutical monodisperse microbubble composition having a composition volume;
a sealed container having an internal container volume in which the stable pharmaceutical monodisperse microbubble composition is disposed, wherein the composition volume is substantially equal to the internal container volume.

35.-42. (canceled)
